(19) United States
(12) Patent Application Publication
(10) Pub. No.: US 2010/0227001 A1
(43) Pub. Date: Sep. 9, 2010

(54) METHOD OF DELIVERING NUTRIENTS

(76) Inventor: SILVIA DEMETER, New York, NY (US)

Correspondence Address: LADAS & PARRY LLP, 26 WEST 61ST STREET, NEW YORK, NY, 10023, US

(21) Appl. No.: 12/398,222

(22) Filed: Mar. 5, 2009

(51) Int. Cl. A61K 33/24; A61K 33/34; A61K 33/32; A61K 33/30; A61K 33/26; A61K 31/122

(52) U.S. Cl. 424/630; 424/639; 424/643; 424/646; 424/655; 514/680

(57) ABSTRACT

A method of delivering nutrients for bariatric patients comprising the sublingual administration of a B-complex vitamin composition and ingesting a liquid, water-soluble multi-vitamin complex orally at approximately the same time. The liquid multi-vitamin is comprised of a plurality of compounds from the group of Vitamin A, Vitamin C, Vitamin D and Vitamin E. After administering the Vitamin B complex and the liquid multi-vitamin, a solid vitamin formulation comprising at least two compounds from the group of iron, zinc, selenium, copper, chromium, Vitamin K, and manganese is ingested orally. A protein solution comprising proteins broken down into small peptide chains, calcium, and fiber may also be delivered orally to the bariatric patient in a liquid form. The method may be repeated once or twice daily. The method of the invention maximizes delivery and absorption of nutrients to the bariatric patient and the tolerance and speed of delivery.

### FIELD OF THE INVENTION

This invention relates generally to methods of delivering nutrients to individuals, and more particularly to individuals that have undergone bariatric surgery.

### BACKGROUND OF THE INVENTION

Bariatric surgery refers to various surgical procedures used to treat obesity that involve the modification of the patient's gastrointestinal tract in order reduce the quantity of nutrients taken in and absorbed. Approximately 170,000 individuals undergo bariatric surgery in the U.S. every year. Primarily restrictive procedures are performed to reduce the patient's stomach size. For instance, laparoscopic adjustable gastric banding is a primarily restrictive bariatric procedure in which a band or belt is placed around the upper part of the stomach. The effective size of the stomach is decreased and leads to a decrease in hunger and the amount of food capable of being comfortably ingested.

Other types of bariatric surgery procedures create a malabsorptive state. For instance, a “biliopancreatic diversion with duodenal switch” switches the duodenum with the ileum as the immediate connection of the stomach to the small intestine. This creates a shorter distance for nutrient absorption time before the food passes to the large intestines.

Gastric bypass surgery such as Roux-en-Y gastric bypass surgery is the most commonly performed bariatric surgery. Roux-en-Y gastric bypass surgery involves both the reduction of the stomach size and the creation of a malabsorptive state. In Roux-en-Y gastric bypass surgery, the stomach area is reduced by creating a small stomach pouch with a stapler device to reduce the stomach by approximately 95%. The size of the new stomach pouch is only able to accommodate 10-15 ml of volume. The malabsorptive state is created in a Roux-en-Y gastric bypass surgery by connecting the small stomach pouch with the distal small intestine. The upper part of the small intestine is then reattached in a Y-shaped configuration.

While bariatric surgery is effective at allowing an individual to lose weight, malnutrition and micronutrient deficiency are known complications of such surgery. The modifications to the stomach in Roux-en-Y gastric bypass surgery and the biliopancreatic diversion with duodenal switch limit the patients' ability to absorb vitamins and nutrients. In addition, the reduction of food consumed by the bariatric patient further reduces the amount of available nutrients. Bariatric patients thus have an increased risk of nutrient deficiencies, such as B-12 deficiency, which can cause anemia and damage to the nervous system.

Accordingly, it is recommended that bariatric patients take vitamin and mineral supplements to ensure that they receive sufficient nutrients. However, as a result of their surgeries, bariatric patients have increased difficulty in digesting and absorbing nutritional supplements in common forms. For instance, vitamins in pill form take at least four hours to dissolve, and the majority of provided supplements have already passed into the lower intestine of the individual by the time the pill dissolves, thereby limiting the benefits of the supplements. Furthermore, traditional supplements put undue stress on the patient's compromised digestive system. Vitamins in pill forms contain binders, fillers, artificial colors, sweeteners, wax coating and preservatives which may tax the digestive system. The vitamins are large and dense and are difficult to swallow and often many pills are needed to satisfy the recommended intake. This causes inconvenience and difficulty in ingesting the required supplements. Pills have also been known to get stuck in the banded portion of a laparoscopic adjustable gastric banding patient. Vomiting due to reactions to common pill supplements can be dangerous and increases the adverse effects of vitamin deficiencies. Vomiting was found to be a risk factor for the development of Wernicke encephalopathy in bariatric patients.

Vitamin B-12 is particularly difficult to metabolize in patients having a Roux-en-Y gastric bypass or a biliopancreatic diversion with duodenal switch procedures because intrinsic factor, a protein produced by glands in the stomach lining, is necessary for full absorption of the Vitamin B complex. Since the stomach is substantially eliminated in these patients, Vitamin B-12 is not metabolized properly. A vitamin B deficiency can lead to further nutrient deficiencies as well; complete absorption of the vitamin B complex is essential for absorption of other nutrients.

Accordingly, it is an object of the present invention to provide a method for nutrient delivery that results in increased absorption of nutrients by bariatric patients.

Yet another object of the present invention is to provide a method for nutrient delivery that is quickly absorbed by bariatric patients.

It is another object of the present invention to provide a method for nutrient delivery that is more easily tolerated by bariatric patients.

Still a further object of the present invention to provide a method for nutrient delivery that is easily administered by bariatric patients.

It is also an object of the present invention to provide a method for nutrient delivery that is efficient and cost effective to implement.

### SUMMARY OF THE INVENTION

The foregoing objects are met by the present invention directed to an improved method of delivering nutrients for bariatric patients. The invention is a multi-step administration regimen, each step being tailored to the specific nutrients to be delivered.

A first step of the method involves the sublingual administration of a B-complex vitamin composition. Sublingual administration provides for a quick and high rate of absorption and avoids the need for processing by the patient's digestive system.

The second step, which may be performed at approximately the same time that the user delivers the Vitamin B composition to the sublingual area, is to ingest a liquid multi-vitamin complex orally. The liquid multi-vitamin is water-soluble and is comprised preferably of two or more of Vitamin A, Vitamin C, Vitamin D and Vitamin E.

After administering the Vitamin B-complex and the liquid multi-vitamin, the user orally ingests a solid vitamin formulation comprising at least two nutrients from the group of Iron, Zinc, Selenium, Copper, Chromium, Vitamin K, and Manganese. The solid multi-vitamin does not contain Vitamin B, which bariatric patients are unable to digest in the stomach. The elimination of Vitamin B differs from other multi-vitamin tabs in the prior art and allows the user to ingest a plurality of vitamins without experiencing digestive problems arising from indigestible B Vitamin compounds.

The combined administration of the sublingual B-complex vitamin composition, the liquid multi-vitamin complex and the solid multi-vitamin as specified in the present invention is so effective that it may be performed only once a day. However, the administration may performed twice daily, if necessary.

In a further embodiment, a protein solution comprising proteins broken down into dipeptide or tripeptide chains is also orally delivered in a liquid form. Calcium and fiber such as Fibersol, a digestive resistant, highly soluble maltodextrin, may also be added to the protein solution.

The method of the invention ensures sufficient nutrient delivery to the bariatric patient while maximizing absorption and speed of delivery. The method substantially limits the ingredients to nutrients that are needed by the bariatric patient and avoids unnecessary ingredients that can bind together with vital nutrients or have an antagonistic effect on such nutrients. The form of nutrients allows a bariatric patient to experience greater tolerance to the delivery of the nutrients and the patient is able to conveniently administer delivery of the nutrients.

### DETAILED DESCRIPTION OF THE PREFERRED EMBODIMENTS

The present invention is directed to an improved method for nutrient delivery, and in particular, for delivering nutrients to bariatric patients. The first step of the method involves the sublingual administration of a B-complex vitamin composition. Sublingual delivery refers to administering substances by bringing them into contact with the highly vascularized mucous membrane, the buccal mucosa, under the tongue. The chemicals diffuse through the buccal mucosa and enter the venous circulation.

The advantages of sublingual delivery as compared with oral administration are that the substances are absorbed much more quickly because they directly enter the bloodstream. Sublingual administration also eliminates processing by the digestive system and avoids degradation from the gastrointestinal tract and liver. For instance, the sublingual administration of the Vitamin B Complex allows a bariatric patient to fully absorb the Vitamin B Complex without requiring the stomach, which lacks intrinsic factor, to process the compound for absorption. There is also a substantially greater degree of absorption from sublingual administration as opposed to oral administration.

The Vitamin B Complex preferably comprises Vitamin B12. The Vitamin B12 form may be cyanocobalamin, a commonly used B12 vitamin form. Vitamin B12 is tolerated at high doses by bariatric patients. The Vitamin B Complex may also comprise Vitamin B6 and Vitamin B9 (folic acid). Both Vitamin B6 and Vitamin B9 must be taken in moderate levels since these components may be harmful at high levels. The preferable concentrations of the Vitamin B Complex are a range of 5-10 mg of Vitamin B6, 1000-2000 mcg of Vitamin B12 and 400-1000 mcg of Vitamin B9.

The sublingual delivery may be accomplished by using a liquid form of the Vitamin B Complex and delivering it to the sublingual area by an appropriately calibrated spray bottle dispenser. Alternatively, a dropper or other means well known in the art may be implemented.

At approximately the same time that the user delivers the Vitamin B composition to the sublingual area, the user orally ingests a liquid multi-vitamin complex. The liquid multi-vitamin is comprised preferably of two or more compounds from the group of Vitamin A, Vitamin C, Vitamin D and Vitamin E. In order to enhance absorption, it is preferable that the vitamins are all in a water soluble form. While Vitamin E is a fat soluble vitamin, Vitamin E may be rendered water soluble by adding a solubizer such as a polysorbate to the compound during the manufacturing process. The water-soluble Vitamin E exhibits increased absorption through the intestinal wall as compared to conventional Vitamin E. The liquid multi-vitamin may also include acerola cherry extract, aloe vera extract, citrus bioflavinoids, grape seed extract, green tea extract, lutein, lycopene, and a trace mineral complex.

Liquid multivitamins ingested orally are more rapidly absorbed by the body and have a significantly higher absorption rate as compared with ingesting a multivitamin in pill or tablet form. Research suggests that only 10-20% of vitamins and minerals in a pill are absorbed by the body compared to 98% for liquid vitamins. The liquid vitamins bypass the digestive process and are absorbed directly into the blood stream. Tests show that 85-90% of the nutrients in a liquid vitamin are absorbed in 25 to 30 seconds. The liquid form is also easier to ingest and is less likely to cause nausea or vomiting than pills. Liquid multi-vitamins also do not contain fillers or other additives which may inhibit absorption or increase the difficulty in metabolizing the nutrients.

A preferable concentration of the liquid multi-vitamins is Vitamin A in the range of 10,000-20,000 IU, Vitamin C in the range of 150-500 IU, Vitamin D in the range of 600-1200 IU, and Vitamin E in the range of 100-400 IU. It is preferred that the liquid multi-vitamin does not contain any Vitamin B compounds, since the Vitamin B Complex is being administered sublingually at the same time as the liquid multi-vitamin is ingested.

After administering the Vitamin B Complex and the liquid multi-vitamin, the user ingests orally a solid vitamin formulation comprising at least two compounds from the group of Iron, Zinc, Selenium, Copper, Chromium, Vitamin K, and Manganese. The preferable concentration of the solid vitamin formulation is iron in the form of Ferronyl® iron in the range of 20-50 mg, Zinc in the form of Zinc Oxide in the range of 20-40 mg, Selenium in the form of Selenomethionine in the range of 70-160 mcg, Copper in the form of Copper Sulfate in the range of 60-150 mcg, Vitamin K in the range of 60 mcg-120 mcg, Chromium in the form of Chromium Chloride in the range of 60-80 mcg, and Manganese in the form of Manganese Sulfate in the range of 2-8 mg. Sufficient delivery and absorption of iron is particular important to bariatric patients because they experience increased difficulty tolerating foods rich in iron such as meat and their compromised digestive system has problems absorbing iron. The multi-vitamin tab preferably has no Vitamin B compounds since bariatric patients have problems digesting such compounds through oral administration.

In a preferred embodiment, the solid multi-vitamin is in the form of a rapid disintegrating tab. These tabs dissolve within seconds of ingestion and offers maximal absorption. The tabs are absorbed in approximately two minutes which is much quicker than multi-vitamins in a typical solid pill form and the tabs are much more easily tolerated than solid pills.

The method of administering the sublingual B-complex vitamin composition, the liquid multi-vitamin complex and the solid multi-vitamin may be performed once or twice daily as needed by the patient.

In another embodiment, a protein solution comprising proteins broken down into dipeptide or tripeptide chains are also orally delivered to the bariatric patient in a liquid form. The small peptide chains are easily digested by the bariatric patient and overcomes the difficulties many patients have metabolizing large protein complexes. It is preferred that the protein solution is made with endoprotease which cuts the protein chains at the proline amino acid to substantially remove bitterness in the protein solution. Calcium and fiber such as Fibersol, a digestive resistant, highly soluble maltodextrin may also be added to the protein solution. In a preferred embodiment the concentration of protein is 15 g, calcium is 60 mg and fiber is 2 g. If the protein solution contains calcium, it is preferable that the protein solution is taken at a different time than the solid multi-vitamin formulation to avoid a mutually antagonistic effect between iron and calcium.

The method of the invention maximizes nutrient delivery to the bariatric patient as well as maximizing absorption and speed of delivery. The form of nutrients allows a bariatric patient to have greater tolerance and the patient is able to conveniently administer delivery of the nutrients. The method substantially limits the ingredients to nutrients that are needed by the bariatric patient and avoids unnecessary ingredients that can bind together with vital nutrients or have an antagonistic effect on an important nutrient.

It should be understood, of course, that the specific form of the invention herein illustrated and described is intended to be representative only, as certain changes may be made therein without departing from the clear teachings of the disclosure. Accordingly, reference should be made to the following appended claims in determining the full scope of the invention.

What is claimed is:

1. An improved method for delivering nutrients to bariatric patients comprising:
administering a liquid B-complex vitamin composition sublingually;
ingesting a liquid multi-vitamin complex orally; and
ingesting orally a multi-vitamin in a solid form comprising two or more compounds from the group of iron, zinc, selenium, copper, chromium, Vitamin K, and manganese.

2. The improved method for delivering nutrients to bariatric patients in claim 1 wherein said sublingual B-complex vitamin composition comprises Vitamin B12.

3. The improved method for delivering nutrients to bariatric patients in claim 2 wherein said sublingual B-complex vitamin comprises 12 mg of Vitamin B12.

4. The improved method for delivering nutrients to bariatric patients in claim 2 wherein said sublingual B-complex vitamin composition further comprises Vitamin B6 and Vitamin B9.

5. The improved method for delivering nutrients to bariatric patients in claim 4 wherein said sublingual B-complex vitamin composition comprises Vitamin B6 in the range of 5-10 mg, Vitamin B12 in the range of 1000-2000 mcg, and Vitamin B9 in the range of 400-1000 mcg.

6. The improved method for delivering nutrients to bariatric patients in claim 4 wherein said sublingual B-complex vitamin composition further comprises thiamin in the range of 1 mg to 2 mg.

7. The improved method for delivering nutrients to bariatric patients in claim 1 wherein said liquid multi-vitamin complex comprises a plurality of compounds from the group of Vitamin A, Vitamin C, Vitamin D and Vitamin E.

8. The improved method for delivering nutrients to bariatric patients in claim 1 wherein said liquid multi-vitamin complex is in a water soluble form.

9. The improved method for delivering nutrients to bariatric patients in claim 7 wherein said liquid multi-vitamin complex comprises a plurality of compounds from said group in the following concentrations: Vitamin A in the range of 10,000-20,000 IU, Vitamin C in the range of 150-500 IU, Vitamin D in the range of 600-1200 IU, and Vitamin E in the range of 100-400 IU.

10. The improved method for delivering nutrients to bariatric patients in claim 1 wherein said solid multi-vitamin comprises a plurality of compounds from said group in the following concentrations: zinc in the range of 20-40 mg, selenium in the range of 70-160 mcg, copper in the range of 60-150 mcg, chromium in the range of 60-80 mcg, manganese in the range of 2-8 mg, and iron in the range of 20-50 mg.

11. The improved method for delivering nutrients to bariatric patients in claim 1 wherein said solid multi-vitamin does not contain Vitamin B.

12. The improved method for delivering nutrients to bariatric patients in claim 1 wherein said steps are repeated once or twice daily.

13. The improved method for delivering nutrients to bariatric patients in claim 1 further comprising the step of ingesting orally a liquid protein solution comprising dipeptides and tripeptides.

14. The improved method for delivering nutrients to bariatric patients in claim 13 wherein said liquid protein solution is manufactured with endoprotease thereby minimizing bitterness in said solution.

15. The improved method for delivering nutrients to bariatric patients in claim 13 wherein said protein solution further comprises calcium.

16. The improved method for delivering nutrients to bariatric patients in claim 15 wherein said protein solution further comprises fiber.

17. The improved method for delivering nutrients to bariatric patients in claim 16 wherein said protein solution comprises 10-20 gm of protein, 45-75 mg of calcium, and 1-3 grams of fiber.

18. The improved method for delivering nutrients to bariatric patients in claim 13 wherein said protein solution is ingested at a different time than said solid multi-vitamin composition.

19. The improved method for delivering nutrients to bariatric patients in claim 1 wherein said solid multi-vitamin is in the form of a rapidly dissolving tab.

20. An improved method for delivering nutrients to bariatric patients comprising:
Administering a liquid B-complex vitamin composition sublingually; and
Ingesting a multi-vitamin complex orally.
